(19) United States
(12) Patent Application Publication
(10) Pub. No.: US 2021/0321907 A1
(43) Pub. Date: Oct. 21, 2021

(54) INTRAVAGINAL EJACULATION LATENCY TIME AND VAGINAL CLONUS MONITORING DEVICE

(71) Applicant: CHENGDU TAN GAO MEDICAL TECHNOLOGY CO., LTD., Chengdu, Sichuan (CN)

(72) Inventors: Jiuhong YUAN, Chengdu, Sichuan (CN); Feng QIN, Chengdu, Sichuan (CN)

(21) Appl. No.: 17/269,124

(22) PCT Filed: Jul. 30, 2019
(86) PCT No.: PCT/CN2019/098362

§ 371 Date: Feb. 17, 2021

(30) Foreign Application Priority Data
Aug. 17, 2018 (CN) 201810943219.3

(51) Int. Cl. A61B 5/11; A61B 5/00

(52) U.S. Cl. CPC A61B 5/1107; A61B 5/4337; A61B 5/4375

(57) ABSTRACT

An intravaginal ejaculation latency time and vaginal clonus monitoring device has a monitoring sleeve, an integrated sensor set and a processing circuit integrated module. The integrated sensor set is located in the monitoring sleeve. The processing circuit integrated module is detachably connected with the monitoring sleeve, and has a real-time clock chip, a central processing unit, a data transmission component and a power module. The integrated sensor set, the real-time clock chip and the data transmission component are all connected with the central processing unit. The power module is used for providing a working power supply.

### TECHNICAL FIELD

The present invention relates to the field of medical monitoring equipment, and in particular relates to an intravaginal ejaculation latency time and vaginal clonus monitoring device.

### BACKGROUND

Premature ejaculation is the most common sexual dysfunction disease in adult men. Premature ejaculation can be defined by two different methods, an objective criterion and subjective perception. The objective criterion definition is an objective index for measuring whether a premature ejaculation symptom exists or not according to the duration from the time when a penis enters the vagina to the time when the penis ejaculates, namely intravaginal ejaculation latency time (IELT). Definition of subjective perception refers to the situation that a man ejaculates before the time he or his partner expects, or feels reduction in ejaculation control, or dissatisfaction or interpersonal communication disorders caused by such situation. The incidence rate of premature ejaculation in the adult men is about 30%, and premature ejaculation severely affects family life and working quality of patients.

At present, preliminary judgment of premature ejaculation mainly relies on inquiring medical history of the patient in the form of evaluation questionnaires and investigating sexual life of the patient. The questionnaires comprise a premature ejaculation diagnosis tool, an Arabian premature ejaculation index, a Chinese premature ejaculation patient sexual function evaluation table and the like. However, based on the various questionnaires, the clinical diagnosis of premature ejaculation mainly depends on chief complaints of the patient. Through intercourse time described by the patient, whether there is premature ejaculation or not is evaluated according to the premature ejaculation evaluation questionnaires, which lacks a practical objective quantitative index. Currently, the use of a chronograph (also known as Stopwatch) to determine the IELT, although objective, requires the patient to manually record start and end times during intercourse, which affects interaction between both man and woman, especially brings great pressure to the man. Therefore, Stopwatch is not clinically practical except for study. In clinical practice, more subjective descriptions and questionnaires are adopted, and support of long-term objective IELT data is lacked.

### SUMMARY

The invention aims to provide an intravaginal ejaculation latency time and vaginal clonus monitoring device which can monitor intravaginal intercourse time, intravaginal twitch amplitude, twitch strength, vaginal clonus and the like. By recording and analyzing the data, scientific data are provided for a patient to assess own sexual function and for doctors to diagnose and treat the patient.

In order to achieve the purpose mentioned above, the present invention is realized by adopting the following technical solution:

the intravaginal ejaculation latency time and vaginal clonus monitoring device provided by the present invention comprises a monitoring sleeve, an integrated sensor set and a processing circuit integrated module, wherein the integrated sensor set is located in the monitoring sleeve (or located in vitro, which can be regarded as two patents); the processing circuit integrated module is detachably connected with the monitoring sleeve; the processing circuit integrated module comprises a real-time clock chip, a central processing unit, a data transmission component and a power module; the integrated sensor set, the real-time clock chip and the data transmission component are all connected with the central processing unit; and the power module is used for providing a working power supply.

Preferably, the integrated sensor set comprises a photoelectric sensor, a piezoelectric sensor, an ultrasonic sensor, a resistance sensor and an electromagnetic sensor; the integrated sensor set is packaged in a cuboid mechanism; the long edge of the cuboid mechanism is in the length direction of the monitoring sleeve; and the monitoring sleeve is provided with an interlayer, and the integrated sensor set is located in the interlayer.

Preferably, the processing circuit integrated module is of a semi-cylindrical arc-shaped structure in appearance, the diameter of the semi-cylindrical arc-shaped structure is larger than that of an opening of the monitoring sleeve, and the end surface of the semi-cylindrical arc-shaped structure is detachably connected with the opening part of the monitoring sleeve.

Furthermore, the device provided by the present invention further comprises an intelligent terminal, the data transmission component is a wireless communication module, and the intelligent terminal is wirelessly connected with the wireless communication module.

Preferably, the wireless communication module is Bluetooth.

Furthermore, the processing circuit integrated module further comprises a data storage device, and the data storage device is connected with the data transmission component and the central processing unit.

Preferably, the integrated sensor set is connected with the central processing unit through an SPI bus or an I²C bus.

Furthermore, the power module comprises a rechargeable button cell, a USB charging socket and a power switch.

Furthermore, a graphical APP is solidified in the intelligent terminal, and the graphical APP is used for analyzing and processing data of the integrated sensor set to obtain total intercourse time, effective intercourse time, an effective intercourse amplitude and an average intercourse amplitude, and automatically generating visual report forms, curve charts and brief summaries.

Furthermore, the device provided by the present invention further comprises an expansion ring connected between the monitoring sleeve and the semi-cylindrical arc-shaped structure.

The working principle of the device provided by the present invention is as follows: the integrated sensor set is arranged in the monitoring sleeve, different signal values measured by the integrated sensor set in an intercourse process are converted into corresponding digital signals to be input into the central processing unit; after the central processing unit analyzes and processes the signals, data are stored in the data storage device for later analysis and processing; and therefore, intercourse twitch amplitude and intercourse twitch strength can be obtained. At the start and end of intercourse, total intravaginal ejaculation latency time (IELT) can be obtained by obtaining the time difference of the real-time clock chip. A monitoring result is transmitted to the intelligent terminal through the Bluetooth; and the data storage device can also copy the data to the intelligent terminal through a data interface for analysis, processing, graphical display and the like.

The intravaginal ejaculation latency time and vaginal clonus monitoring device provided by the present invention has the following beneficial effects:
1, the device can automatically record the ejaculation latency time, intercourse twitch amplitude, intercourse twitch strength, intercourse twitch frequency and other data, without affecting interaction between both man and woman during intercourse.
2, the device is simple and convenient to use.
3, the device utilizes various sensors for recording in real time, the recorded data are objective and reliable, and data support is provided for diagnosis of the doctors.
4, the processing circuit integrated module of the device adopts a detachable and waterproof design, can be charged in various modes, can be repeatedly used, and is relatively low in cost.
5, the data can be received and stored by using an application, and the application is installed in an existing computer or mobile phone, so that hardware costs are reduced.

### BRIEF DESCRIPTION OF THE DRAWINGS

FIG. 1 is a schematic diagram of a monitoring sleeve and an integrated sensor set of the present invention; and

FIG. 2 is a schematic diagram of a processing circuit integrated module of the present invention.

In the FIGURES: 1—monitoring sleeve, 2—integrated sensor set, 3—processing circuit integrated module, 4—expansion ring, 5—switch and indicating lamp part, 6—data transmission module, 7—sensor circuit interface and 8—buckle.

### DETAILED DESCRIPTION

In order to make objectives, technical solutions and advantages of the present invention clearer, the present invention is described in further detail below in combination with accompanying drawings.

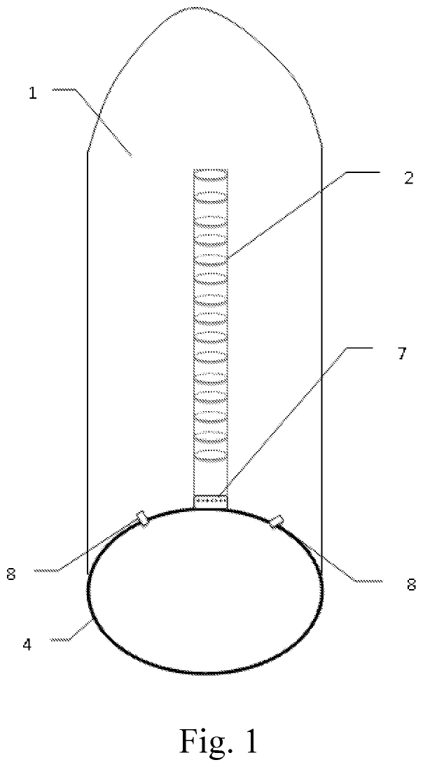

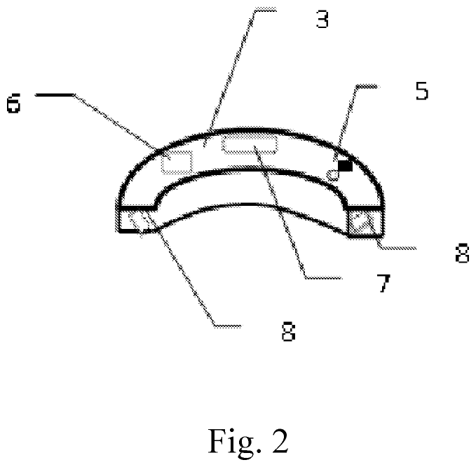

As shown in FIG. 1 and FIG. 2, the intravaginal ejaculation latency time and vaginal clonus monitoring device comprises a monitoring sleeve 1, an integrated sensor set 2, a processing circuit integrated module 3, a data storage device, an application and an intelligent terminal, wherein the integrated sensor set 2 and the processing circuit integrated module 3 are both installed on the monitoring sleeve 1; the processing circuit integrated module 3 is connected with the integrated sensor set 2 and the data storage device; the integrated sensor set 2 is used for monitoring intercourse frequency and amplitude; the processing circuit integrated module 3 is used for receiving and processing an output signal of the integrated sensor set, transmitting information to the intelligent terminal through the application and receiving a control signal of the intelligent terminal; and the intelligent terminal is used for analyzing the received information to obtain related parameters of sexual capacity.

The processing circuit integrated module 3 and the data storage device are fixed to the position close to an opening of the monitoring sleeve 1 through buckles 8, and the four buckles 8 are installed on an expansion ring 4 and the processing circuit integrated module 3 in pairs and correspond to the root position of the penis of a human body. The processing circuit integrated module 3 comprises a real-time clock chip, a central processing unit, a wireless communication module, a power module, a switch and indicating lamp part 5, a data transmission module 6 and a sensor circuit interface 7.

The integrated sensor set 2 is arranged on the monitoring sleeve 1 in the axial direction of the monitoring sleeve 1; the integrated sensor set 2 comprises a photoelectric sensor, a piezoelectric sensor, an ultrasonic sensor, a resistance sensor, an electromagnetic sensor and a sensor circuit interface 7; and the sensor circuit interface 7 of the integrated sensor set 2 is connected with the sensor circuit interface 7 of the processing circuit integrated module 3.

When in actual use, an examinee wears the monitoring sleeve 1 in a traditional condom wearing mode at first, the processing circuit integrated module 3 is installed on the expansion ring 4 after the monitoring sleeve 1 is worn, the sensor circuit interface 7 of the processing circuit integrated module 3 is connected with the sensor circuit interface 7 of the integrated sensor set 2, and after the switch and indicating lamp part 5 is turned on, normal intercourse is carried out; real-time information is monitored through the integrated sensor set 2 and transmitted to the central processing unit, the central processing unit analyzes and processes the real-time information and then stores the real-time information into the data storage device, the real-time clock chip records specific start and end times, and therefore the data such as total ejaculation latency time, intercourse twitch amplitude, intercourse twitch strength, intercourse twitch frequency and vaginal clonus can be obtained. After intercourse is finished, a power switch is turned off, and the processing circuit integrated module 3 is taken down. In addition, the data in the data storage device can be transmitted to the intelligent terminal through the application, and a series of visual reports, curve charts, brief summaries and the like can be automatically generated by analyzing the obtained data.

Of course, the present invention can also have other various embodiments, and various corresponding changes and modifications can be made in light of the present invention by those skilled in the art without departing from the spirit and essence of the present invention, but these changes and modifications should fall within the scope of the claims appended hereto.

1. An intravaginal ejaculation latency time and vaginal clonus monitoring device, characterized by comprising a monitoring sleeve, an integrated sensor set and a processing circuit integrated module, wherein the integrated sensor set is located in the monitoring sleeve; the processing circuit integrated module is detachably connected with the monitoring sleeve; the processing circuit integrated module comprises a real-time clock chip, a central processing unit, a data transmission component and a power module; the integrated sensor set, the real-time clock chip and the data transmission component are all connected with the central processing unit; and the power module is used for providing a working power supply.

2. The intravaginal ejaculation latency time and vaginal clonus monitoring device according to claim 1, characterized in that the integrated sensor set comprises a photoelectric sensor, a piezoelectric sensor, an ultrasonic sensor, a resistance sensor and an electromagnetic sensor; the integrated sensor set is packaged in a cuboid mechanism; the long edge of the cuboid mechanism is in the length direction of the monitoring sleeve; the monitoring sleeve is provided with an interlayer; and the integrated sensor set is located in the interlayer.

3. The intravaginal ejaculation latency time and vaginal clonus monitoring device according to claim 2, characterized in that the processing circuit integrated module is of a semi-cylindrical arc-shaped structure in appearance, the diameter of the semi-cylindrical arc-shaped structure is larger than that of an opening of the monitoring sleeve, and the end surface of the semi-cylindrical arc-shaped structure is detachably connected with the opening part of the monitoring sleeve.

4. The intravaginal ejaculation latency time and vaginal clonus monitoring device according to claim 1, characterized by further comprising an intelligent terminal, wherein the data transmission component is a wireless communication module, and the intelligent terminal is wirelessly connected with the wireless communication module.

5. The intravaginal ejaculation latency time and vaginal clonus monitoring device according to claim 4, characterized in that the wireless communication module is Bluetooth.

6. The intravaginal ejaculation latency time and vaginal clonus monitoring device according to claim 4, characterized in that the processing circuit integrated module further comprises a data storage device, and the data storage device is connected with the data transmission component and the central processing unit.

7. The intravaginal ejaculation latency time and vaginal clonus monitoring device according to claim 4, characterized in that the integrated sensor set is connected with the central processing unit through an SPI bus or an I²C bus.

8. The intravaginal ejaculation latency time and vaginal clonus monitoring device according to claim 4, characterized in that the power module comprises a rechargeable button cell, a USB charging socket and a power switch.

9. The intravaginal ejaculation latency time and vaginal clonus monitoring device according to claim 4, characterized in that a graphical APP is solidified in the intelligent terminal, and the graphical APP is used for analyzing and processing data of the integrated sensor set to obtain total intercourse time, effective intercourse time, an effective intercourse amplitude and an average intercourse amplitude, and automatically generating visual report forms, curve charts and brief summaries.

10. The intravaginal ejaculation latency time and vaginal clonus monitoring device according to claim 3, characterized by further comprising an expansion ring connected between the monitoring sleeve and the semi-cylindrical arc-shaped structure.
